(19) United States
(12) Patent Application Publication
(10) Pub. No.: US 2005/0003483 A1
(43) Pub. Date: Jan. 6, 2005

(54) Comparative ligand mapping from MHC class 1 positive cells

(76) Inventors: William H. Hildebrand, Edmond, OK (US); Heather D. Hickman-Miller, Rockville, MD (US)

Correspondence Address: DUNLAP, CODDING & ROGERS P.C., PO BOX 16370, OKLAHOMA CITY, OK, 73113, US

(21) Appl. No.: 10/845,391

(22) Filed: May 13, 2004

Related U.S. Application Data
(63) Continuation-in-part of application No. 09/974,366, filed on Oct. 10, 2001.
(60) Provisional application No. 60/469,995, filed on May 13, 2003.
(60) Provisional application No. 60/518,132, filed on Nov. 7, 2003.
(60) Provisional application No. 60/240,143, filed on Oct. 10, 2000.
(60) Provisional application No. 60/299,452, filed on Jun. 20, 2001.
(60) Provisional application No. 60/256,410, filed on Dec. 18, 2000.
(60) Provisional application No. 60/256,409, filed on Dec. 18, 2000.
(60) Provisional application No. 60/327,907, filed on Oct. 9, 2001.

(51) Int. Cl. C12P 21/02; C07H 21/04; C07K 16/18

(52) U.S. Cl. 435/69.1; 435/320.1; 435/326; 530/388.1; 536/23.53

(57) ABSTRACT

The present invention relates generally to a methodology for the isolation, purification and identification of peptide ligands presented by MHC positive cells. In particular, the methodology of the present invention relates to the isolation, purification and identification of these peptide ligands from soluble class I and class II MHC molecules which may be from uninfected, infected, or tumorigenic cells. The methodology of the present invention broadly allows for these peptide ligands and their cognate source proteins thereof to be identified and used as markers for infected versus uninfected cells and/or tumorigenic versus nontumorigenic cells with said identification being useful for marking or targeting a cell for therapeutic treatment or priming the immune response against infected cells.

### CROSS-REFERENCE TO RELATED APPLICATIONS

This application claims the benefit under 35 U.S.C. 119(e) of provisional applications U.S. Ser. No. 60/469,995, filed May 13, 2003, and U.S. Ser. No. 60/518,132, filed Nov. 7, 2003, the contents of each of which are hereby expressly incorporated herein by reference in their entirety.

This application is also a continuation-in-part of U.S. Ser. No. 09/974,366, filed Oct. 10, 2001, which claims the benefit under 35 U.S.C. 119(e) of provisional applications U.S. Ser. No. 60/240,143, filed Oct. 10, 2000; U.S. Ser. No. 60/299,452, filed Jun. 20, 2001; U.S. Ser. No. 60/256,410, filed Dec. 18, 2000; U.S. Ser. No. 60/256,409, filed Dec. 18, 2000; and U.S. Ser. No. 60/327,907, filed Oct. 9, 2001; all of which are hereby expressly incorporated herein by reference in their entirety.

### STATEMENT REGARDING FEDERALLY SPONSORED RESEARCH OR DEVELOPMENT

Not applicable.

### BACKGROUND OF THE INVENTION

1. Field of the Invention

The present invention relates generally to a methodology of epitope testing for the identification of peptides that bind to an individual soluble MHC Class I or Class II molecule as well as to peptides identified by such methodology.

2. Description of the Background Art

Class I major histocompatibility complex (MHC) molecules, designated HLA class I in humans, bind and display peptide antigen ligands upon the cell surface. The peptide antigen ligands presented by the class I MHC molecule are derived from either normal endogenous proteins (“self”) or foreign proteins (“nonself”) introduced into the cell. Nonself proteins may be products of malignant transformation or intracellular pathogens such as viruses. In this manner, class I MHC molecules convey information regarding the internal fitness of a cell to immune effector cells including but not limited to, CD8⁺ cytotoxic T lymphocytes (CTLs), which are activated upon interaction with “nonself” peptides, thereby lysing or killing the cell presenting such “nonself” peptides.

Class II MHC molecules, designated HLA class II in humans, also bind and display peptide antigen ligands upon the cell surface. Unlike class I MHC molecules which are expressed on virtually all nucleated cells, class II MHC molecules are normally confined to specialized cells, such as B lymphocytes, macrophages, dendritic cells, and other antigen presenting cells which take up foreign antigens from the extracellular fluid via an endocytic pathway. The peptides they bind and present are derived from extracellular foreign antigens, such as products of bacteria that multiply outside of cells, wherein such products include protein toxins secreted by the bacteria that often times have deleterious and even lethal effects on the host (e.g. human). In this manner, class II molecules convey information regarding the fitness of the extracellular space in the vicinity of the cell displaying the class II molecule to immune effector cells, including but not limited to, CD4⁺ helper T cells, thereby helping to eliminate such pathogens the examination of such pathogens is accomplished by both helping B cells make antibodies against microbes, as well as toxins produced by such microbes, and by activating macrophages to destroy ingested microbes.

Class I and class II HLA molecules exhibit extensive polymorphism generated by systematic recombinatorial and point mutation events; as such, hundreds of different HLA types exist throughout the world's population, resulting in a large immunological diversity. Such extensive HLA diversity throughout the population results in tissue or organ transplant rejection between individuals as well as differing susceptibilities and/or resistances to infectious diseases. HLA molecules also contribute significantly to autoimmunity and cancer. Because HLA molecules mediate most, if not all, adaptive immune responses, large quantities of pure isolated HLA proteins are required in order to effectively study transplantation, autoimmunity disorders, and for vaccine development.

There are several applications in which purified, individual class I and class II MHC proteins are highly useful. Such applications include using MHC-peptide multimers as immunodiagnostic reagents for disease resistance/autoimmunity; assessing the binding of potentially therapeutic peptides; elution of peptides from MHC molecules to identify vaccine candidates; screening transplant patients for preformed MHC specific antibodies; and removal of anti-HLA antibodies from a patient. Since every individual has differing MHC molecules, the testing of numerous individual MHC molecules is a prerequisite for understanding the differences in disease susceptibility between individuals. Therefore, purified MHC molecules representative of the hundreds of different HLA types existing throughout the world's population are highly desirable for unraveling disease susceptibilities and resistances, as well as for designing therapeutics such as vaccines.

Class I HLA molecules alert the immune response to disorders within host cells. Peptides, which are derived from viral- and tumor-specific proteins within the cell, are loaded into the class I molecule's antigen binding groove in the endoplasmic reticulum of the cell and subsequently carried to the cell surface. Once the class I HLA molecule and its loaded peptide ligand are on the cell surface, the class I molecule and its peptide ligand are accessible to cytotoxic T lymphocytes (CTL). CTL survey the peptides presented by the class I molecule and destroy those cells harboring ligands derived from infectious or neoplastic agents within that cell.

While specific CTL targets have been identified, little is known about the breadth and nature of ligands presented on the surface of a diseased cell. From a basic science perspective, many outstanding questions have percolated through the art regarding peptide exhibition. For instance, it has been demonstrated that a virus can preferentially block expression of HLA class I molecules from a given locus while leaving expression at other loci intact. Similarly, there are numerous reports of cancerous cells that fail to express class I HLA at particular loci. However, there is no data describing how (or if) the three classical HLA class I loci differ in the immunoregulatory ligands they bind. It is therefore unclear how class I molecules from the different loci vary in their interaction with viral- and tumor-derived ligands and the number of peptides each will present.

Discerning virus- and tumor-specific ligands for CTL recognition is an important component of vaccine design. Ligands unique to tumorigenic or infected cells can be tested and incorporated into vaccines designed to evoke a protective CTL response. Several methodologies are currently employed to identify potentially protective peptide ligands. One approach uses T cell lines or clones to screen for biologically active ligands among chromatographic fractions of eluted peptides (Cox et al., Science, vol 264, 1994, pages 716-719, which is expressly incorporated herein by reference in its entirety). This approach has been employed to identify peptide ligands specific to cancerous cells. A second technique utilizes predictive algorithms to identify peptides capable of binding to a particular class I molecule based upon previously determined motif and/or individual ligand sequences (De Groot et al., Emerging Infectious Diseases, (7) 4, 2001, which is expressly incorporated herein by reference in its entirety). Peptides having high predicted probability of binding from a pathogen of interest can then be synthesized and tested for T cell reactivity in various assays, such as but not limited to, precursor, tetramer and ELISpot assays.

However, there has been no readily available source of individual HLA molecules. The quantities of HLA protein available have been small and typically consist of a mixture of different HLA molecules. Production of HLA molecules traditionally involves growth and lysis of cells expressing multiple HLA molecules. Ninety percent of the population is heterozygous at each of the HLA loci; codominant expression results in multiple HLA proteins expressed at each HLA locus. To purify native class I or class II molecules from mammalian cells requires time-consuming and cumbersome purification methods, and since each cell typically expresses multiple surface-bound HLA class I or class II molecules, HLA purification results in a mixture of many different HLA class I or class II molecules. When performing experiments using such a mixture of HLA molecules or performing experiments using a cell having multiple surface-bound HLA molecules, interpretation of results cannot directly distinguish between the different HLA molecules, and one cannot be certain that any particular HLA molecule is responsible for a given result. Therefore, prior to the present invention, a need existed in the art for a method of producing substantial quantities of individual HLA class I or class II molecules so that they can be readily purified and isolated independent of other HLA class I or class II molecules. Such individual HLA molecules, when provided in sufficient quantity and purity as described herein, provides a powerful tool for studying and measuring immune responses.

Therefore, there exists a need in the art for improved methods of assaying binding of peptides to class I and class II MHC molecules to identify epitopes that bind to specific individual class I and class II MHC molecules. The present invention solves this need by coupling the production of soluble HLA molecules with epitope isolation, discovery, and testing methodology.

### BRIEF DESCRIPTION OF THE DRAWINGS

FIG. 1. Overview of 2 stage PCR strategy to amplify a truncated version of the human class I MHC.

FIG. 2. Flow chart of the epitope discovery of C-terminal-tagged sHLA molecules. Class I positive transfectants are infected with a pathogen of choice, and sHLA is preferentially purified utilizing the tag. Subtractive comparison of MS ion maps yields ions present only in infected cell, which are then MS/MS sequenced to derive class I epitopes.

### DETAILED DESCRIPTION OF THE INVENTION

Before explaining at least one embodiment of the invention in detail by way of exemplary drawings, experimentation, results, and laboratory procedures, it is to be understood that the invention is not limited in its application to the details of construction and the arrangement of the components set forth in the following description or illustrated in the drawings, experimentation and/or results. The invention is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

The present invention combines methodologies for assaying the binding of peptide epitopes to individual, soluble MHC molecules with methodologies for the production of individual, soluble MHC molecules and with a method of epitope discovery and comparative ligand mapping (including methods of distinguishing infected/tumor cells from uninfected/non-tumor cells). The method of production of individual, soluble MHC molecules has previously been described in detail in parent application U.S. Ser. No. 10/022,066, filed Dec. 18, 2001, entitled “METHOD AND APPARATUS FOR THE PRODUCTION OF SOLUBLE MHC ANTIGENS AND USES THEREOF,” the contents of which are hereby expressly incorporated herein in their entirety by reference. The method of epitope discovery and comparative ligand mapping has previously been described in detail in parent application U.S. Ser. No. 09/974,366, filed Oct. 10, 2001, entitled “COMPARATIVE LIGAND MAPPING FROM MHC CLASS I POSITIVE CELLS”, the contents of which have previously been expressly incorporated in their entirety by reference. A brief description of each of these methodologies is included herein below for the purpose of exemplification and should not be considered as limiting.

In addition, the methods of the present invention may be combined with methods of epitope testing as described in U.S. Ser. No. 10/095,818, filed Mar. 11, 2002, entitled “EPITOPE TESTING USING SOLUBLE HLA”, the contents of which are hereby expressly incorporated herein by reference.

To produce the individual soluble class I molecule-endogenous peptide complexes, genomic DNA or cDNA encoding at least one class I molecule is obtained, and an allele encoding an individual class I molecule in the genomic DNA or cDNA is identified. The allele encoding the individual class I molecule is PCR amplified in a locus specific manner such that a PCR product produced therefrom encodes a truncated, soluble form of the individual class I molecule. The PCR product is then cloned into an expression vector, thereby forming a construct that encodes the individual soluble class I molecule, and the construct is transfected into a cell line to provide a cell line containing a construct that encodes an individual soluble class I molecule. The cell line must be able to naturally process proteins into peptide ligands capable of being loaded into antigen binding grooves of class I molecules.

The cell line is then cultured under conditions which allow for expression of the individual soluble class I molecules from the construct, and these conditions also allow for endogenous loading of a peptide ligand into the antigen binding groove of each individual soluble class I molecule prior to secretion of the individual soluble class I molecules from the cell. The secreted individual soluble class I molecules having the endogenously loaded peptide ligands bound thereto are then isolated.

The construct that encodes the individual soluble class I molecule may further encode a tag, such as a HIS tail or a FLAG tail, which is attached to the individual soluble class I molecule and aids in isolating the individual soluble class I molecule.

The peptide of interest may be chosen based on several methods of epitope discovery known in the art. Alternatively, the peptide of interest may be identified by a method for identifying at least one endogenously loaded peptide ligand that distinguishes an infected cell from an uninfected cell. Such method includes providing an uninfected cell line containing a construct that encodes an individual soluble class I molecule, wherein the uninfected cell line is able to naturally process proteins into peptide ligands capable of being loaded into antigen binding grooves of class I molecules. A portion of the uninfected cell line is infected with at least one of a microorganism (such as HIV or HBV), a gene from a microorganism or a tumor gene, thereby providing an infected cell line, and both the uninfected cell line and the infected cell line are cultured under conditions which allow for expression of individual soluble class I molecules from the construct. The culture conditions also allow for endogenous loading of a peptide ligand in the antigen binding groove of each individual soluble class I molecule prior to secretion of the individual soluble class I molecules from the cell. The secreted individual soluble class I molecules having the endogenously loaded peptide ligands bound thereto are isolated from the uninfected cell line and the infected cell line, and the endogenously loaded peptide ligands are separated from the individual soluble class I molecules from both the uninfected cell line and the infected cell line. The endogenously loaded peptide ligands are then isolated from both the uninfected cell line and the infected cell line, and the two sets of endogenously loaded peptide ligands are compared to identify at least one endogenously loaded peptide ligand presented by the individual soluble class I molecule on the infected cell line that is not presented by the individual soluble class I molecule on the uninfected cell line, or to identify at least one endogenously loaded peptide ligand presented by the individual soluble class I molecule on the uninfected cell line that is not presented by the individual soluble class I molecule on the infected cell line.

Optionally, proteomics may eventually allow for sequencing all epitopes from a diseased cell so that comparative mapping, i.e., comparison of infected cells to healthy cells, would no longer be required. Microarrays and other proteomic data should provide insight as to the healthy cell.

Following identification of the peptide ligand that distinguishes an infected cell from an uninfected cell, a source protein from which the endogenously loaded peptide ligand is obtained can be identified. Such source protein may be encoded by at least one of the microorganism, the gene from a microorganism or the tumor gene with which the cell line was infected to form the infected cell line, or the source protein may be encoded by the uninfected cell line. When the source protein is encoded by the uninfected cell line, such protein may also demonstrate increased expression in a tumor cell line.

### Production of Individual, Soluble MHC Molecules

The methods of the present invention may, in one embodiment, utilize a method of producing MHC molecules (from genomic DNA or cDNA) that are secreted from mammalian cells in a bioreactor unit. Substantial quantities of individual MHC molecules are obtained by modifying class I or class II MHC molecules so that they are capable of being secreted, isolated, and purified. Secretion of soluble MHC molecules overcomes the disadvantages and defects of the prior art in relation to the quantity and purity of MHC molecules produced. Problems of quantity are overcome because the cells producing the MHC do not need to be detergent lysed or killed in order to obtain the MHC molecule. In this way the cells producing secreted MHC remain alive and therefore continue to produce MHC. Problems of purity are overcome because the only MHC molecule secreted from the cell is the one that has specifically been constructed to be secreted. Thus, transfection of vectors encoding such secreted MHC molecules into cells which may express endogenous, surface bound MHC provides a method of obtaining a highly concentrated form of the transfected MHC molecule as it is secreted from the cells. Greater purity is assured by transfecting the secreted MHC molecule into MHC deficient cell lines.

Production of the MHC molecules in a hollow fiber bioreactor unit allows cells to be cultured at a density substantially greater than conventional liquid phase tissue culture permits. Dense culturing of cells secreting MHC molecules further amplifies the ability to continuously harvest the transfected MHC molecules. Dense bioreactor cultures of MHC secreting cell lines allow for high concentrations of individual MHC proteins to be obtained. Highly concentrated individual MHC proteins provide an advantage in that most downstream protein purification strategies perform better as the concentration of the protein to be purified increases. Thus, the culturing of MHC secreting cells in bioreactors allows for a continuous production of individual MHC proteins in a concentrated form.

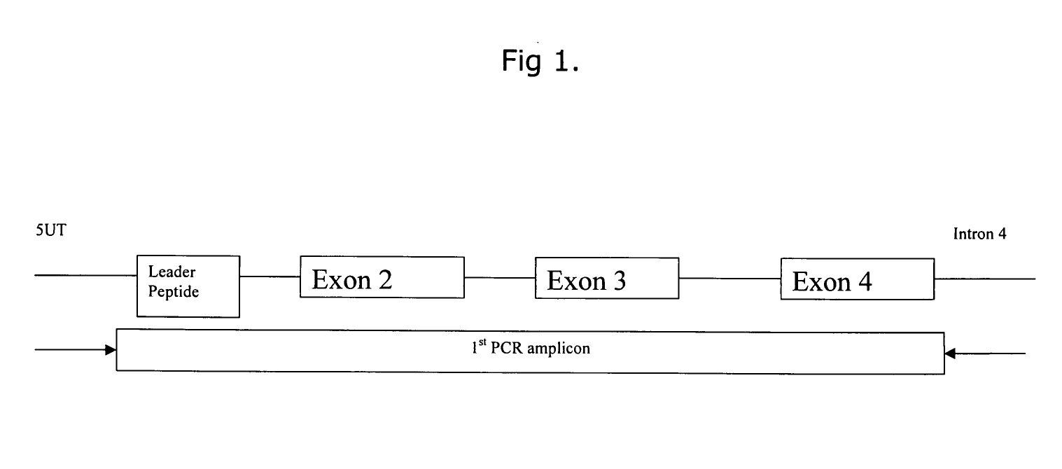

The method of producing MHC molecules utilized in the present invention and described in detail in U.S. Ser. No. 10/022,066 begins by obtaining genomic or complementary DNA which encodes the desired MHC class I or class II molecule. Alleles at the locus which encode the desired MHC molecule are PCR amplified in a locus specific manner. These locus specific PCR products may include the entire coding region of the MHC molecule or a portion thereof. In one embodiment a nested or hemi-nested PCR is applied to produce a truncated form of the class I or class II gene so that it will be secreted rather than anchored to the cell surface. FIG. 1 illustrates the PCR products resulting from such nested PCR reactions. In another embodiment the PCR will directly truncate the MHC molecule.

Locus specific PCR products are cloned into a mammalian expression vector and screened with a variety of methods to identify a clone encoding the desired MHC molecule. The cloned MHC molecules are DNA sequenced to ensure fidelity of the PCR. Faithful truncated clones of the desired MHC molecule are then transfected into a mammalian cell line. When such cell line is transfected with a vector encoding a recombinant class I molecule, such cell line may either lack endogenous class I MHC molecule expression or express endogenous class I MHC molecules. One of ordinary skill of the art would note the importance, given the present invention, that cells expressing endogenous class I MHC molecules may spontaneously release MHC into solution upon natural cell death, infection, transformation, etc. In cases where this small amount of spontaneously released MHC is a concern, the transfected class I MHC molecule can be “tagged” such that it can be specifically purified away from spontaneously released endogenous class I molecules in cells that express class I molecules. For example, a DNA fragment encoding a HIS tail may be attached to the protein by the PCR reaction or may be encoded by the vector into which the PCR fragment is cloned, and such HIS tail, therefore, further aids in the purification of the class I MHC molecules away from endogenous class I molecules. Tags beside a histidine tail have also been demonstrated to work, and one of ordinary skill in the art of tagging proteins for downstream purification would appreciate and know how to tag a MHC molecule in such a manner so as to increase the ease by which the MHC molecule may be purified.

Cloned genomic DNA fragments contain both exons and introns as well as other non-translated regions at the 5′ and 3′ termini of the gene. Following transfection into a cell line which transcribes the genomic DNA (gDNA) into RNA, cloned genomic DNA results in a protein product thereby removing introns and splicing the RNA to form messenger RNA (mRNA), which is then translated into an MHC protein. Transfection of MHC molecules encoded by gDNA therefore facilitates reisolation of the gDNA, mRNA/cDNA, and protein. Production of MHC molecules in non-mammalian cell lines such as insect and bacterial cells requires cDNA clones, as these lower cell types do not have the ability to splice introns out of RNA transcribed from a gDNA clone. In these instances the mammalian gDNA transfectants of the present invention provide a valuable source of RNA which can be reverse transcribed to form MHC cDNA. The cDNA can then be cloned, transferred into cells, and then translated into protein. In addition to producing secreted MHC, such gDNA transfectants therefore provide a ready source of mRNA, and therefore cDNA clones, which can then be transfected into non-mammalian cells for production of MHC. Thus, the present invention which starts with MHC genomic DNA clones allows for the production of MHC in cells from various species.

A key advantage of starting from gDNA is that viable cells containing the MHC molecule of interest are not needed. Since all individuals in the population have a different MHC repertoire, one would need to search more than 500,000 individuals to find someone with the same MHC complement as a desired individual—such a practical example of this principle is observed when trying to find a donor to match a recipient for bone marrow transplantation. Thus, if it is desired to produce a particular MHC molecule for use in an experiment or diagnostic, a person or cell expressing the MHC allele of interest would first need to be identified. Alternatively, in the method of the present invention, only a saliva sample, a hair root, an old freezer sample, or less than a milliliter (0.2 ml) of blood would be required to isolate the gDNA. Then, starting from gDNA, the MHC molecule of interest could be obtained via a gDNA clone as described herein, and following transfection of such clone into mammalian cells, the desired protein could be produced directly in mammalian cells or from cDNA in several species of cells using the methods of the present invention described herein.

Current methodologies used by others to obtain an MHC allele for protein expression typically start from mRNA, which requires a fresh sample of mammalian cells that express the MHC molecule of interest. Working from gDNA does not require gene expression or a fresh biological sample. It is also important to note that RNA is inherently unstable and is not as easily obtained as is gDNA. Therefore, if production of a particular MHC molecule starting from a cDNA clone is desired, a person or cell line that is expressing the allele of interest must traditionally first be identified in order to obtain RNA. Then a fresh sample of blood or cells must be obtained; experiments using the methodology of the present invention show that ≧5 milliliters of blood that is less than 3 days old is required to obtain sufficient RNA for MHC cDNA synthesis. Thus, by starting with gDNA, the breadth of MHC molecules that can be readily produced is expanded. This is a key factor in a system as polymorphic as the MHC system; hundreds of MHC molecules exist, and not all MHC molecules are readily available. This is especially true of MHC molecules unique to isolated populations or of MHC molecules unique to ethnic minorities. Starting class I or class II MHC molecule expression from the point of genomic DNA simplifies the isolation of the gene of interest and insures a more equitable means of producing MHC molecules for study; otherwise, one would be left to determine whose MHC molecules are chosen and not chosen for study, as well as to determine which ethnic population from which fresh samples cannot be obtained and therefore should not have their MHC molecules included in a diagnostic assay.

While CDNA may be substituted for genomic DNA as the starting material, production of CDNA for each of the desired HLA class I types will require hundreds of different, HLA typed, viable cell lines, each expressing a different HLA class I type. Alternatively, fresh samples are required from individuals with the various desired MHC types. The use of genomic DNA as the starting material allows for the production of clones for many HLA molecules from a single genomic DNA sequence, as the amplification process can be manipulated to mimic recombinatorial and gene conversion events. Several mutagenesis strategies exist whereby a given class I gDNA clone could be modified at either the level of gDNA or at the cDNA resulting from this gDNA clone. The process of producing MHC molecules utilized in the present invention does not require viable cells, and therefore the degradation which plagues RNA is not a problem.

### Methods of Epitope Discovery and Comparative Ligand Mapping

Peptide epitopes unique to infected and cancerous cells can be directly identified by the methods of the present invention, which include producing sHLA molecules in cancerous and infected cells and then sequencing the epitopes unique to the cancerous or infected cells. Such epitopes can then be tested for their binding to various HLA molecules to see how many HLA molecules these epitopes might bind. This direct method of epitope discovery is described in detail in U.S. Ser. No. 09/974,366 and is briefly described herein below.

The method of epitope discovery included in the present invention (and described in detail in U.S. Ser. No. 09/974,366) includes the following steps: (1) providing a cell line containing a construct that encodes an individual soluble class I or class II MHC molecule (wherein the cell line is capable of naturally processing self or nonself proteins into peptide ligands capable of being loaded into the antigen binding grooves of the class I or class II MHC molecules); (2) culturing the cell line under conditions which allow for expression of the individual soluble class I or class II MHC molecule from the construct, with such conditions also allowing for the endogenous loading of a peptide ligand (from the self or non-self processed protein) into the antigen binding groove of each individual soluble class I or class II MHC molecule prior to secretion of the soluble class I or class II MHC molecules having the peptide ligands bound thereto; and (3) separating the peptide ligands from the individual soluble class I or class II MHC molecules.

Class I and class II MHC molecules are really a trimolecular complex consisting of an alpha chain, a beta chain, and the alpha/beta chain's peptide cargo (i.e. the peptide ligand) which is presented on the cell surface to immune effector cells. Since it is the peptide cargo, and not the MHC alpha and beta chains, which marks a cell as infected, tumorigenic, or diseased, there is a great need to identify and characterize the peptide ligands bound by particular MHC molecules. For example, characterization of such peptide ligands greatly aids in determining how the peptides presented by a person with MHC-associated diabetes differ from the peptides presented by the MHC molecules associated with resistance to diabetes. As stated above, having a sufficient supply of an individual MHC molecule, and therefore that MHC molecule's bound peptides, provides a means for studying such diseases. Because the method of the present invention provides quantities of MHC protein previously unobtainable, unparalleled studies of MHC molecules and their important peptide cargo can now be facilitated and utilized to distinguish infected/tumor cells from uninfected/non-tumor cells by unique epitopes presented by MHC molecules in the disease or non-disease state.

The method of the present invention includes the direct comparative analysis of peptide ligands eluted from class I HLA molecules (as described previously in U.S. Ser. No. 09/974,366). The teachings of U.S. Ser. No. 09/974,366 demonstrates that the addition of a C-terminal epitope tag (such as a 6-HIS or FLAG tail) to transfected class I molecules has no effects on peptide binding specificity of the class I molecule and consequently has no deleterious effects on direct peptide ligand mapping and sequencing, and also does not disrupt endogenous peptide loading.

The method described in parent application U.S. Ser. No. 09/974,366 further relates to a novel method for detecting those peptide epitopes which distinguish the infected/tumor cell from the uninfected/non-tumor cell. The results obtained from the present inventive methodology cannot be predicted or ascertained indirectly; only with a direct epitope discovery method can the unique epitopes described therein be identified. Furthermore, only with this direct approach can it be ascertained that the source protein is degraded into potentially immunogenic peptide epitopes. Finally, this unique approach provides a glimpse of which proteins are uniquely up and down regulated in infected/tumor cells.

The utility of such HLA-presented peptide epitopes which mark the infected/tumor cell are three-fold. First, diagnostics designed to detect a disease state (i.e., infection or cancer) can use epitopes unique to infected/tumor cells to ascertain the presence/absence of a tumor/virus. Second, epitopes unique to infected/tumor cells represent vaccine candidates. For example, the present invention describes and claims epitopes which arise on the surface of cells infected with HIV. Such epitopes could not be predicted without natural virus infection and direct epitope discovery. The epitopes detected are derived from proteins unique to virus infected and tumor cells. These epitopes can be used for virus/tumor vaccine development and virus/tumor diagnostics. Third, the process indicates that particular proteins unique to virus infected cells are found in compartments of the host cell they would otherwise not be found in. Thus, uniquely upregulated or trafficked host proteins are identified for drug targeting to kill infected cells.

While the epitopes detected as unique to infected/tumor cells may serve as direct targets (i.e., through diagnostic, vaccine or therapeutic means), such epitopes may also be utilized to influence the environment around a diseased cell so that these treatments and therapies are effective, and thus allowing the immune responses to see the diseased cell.

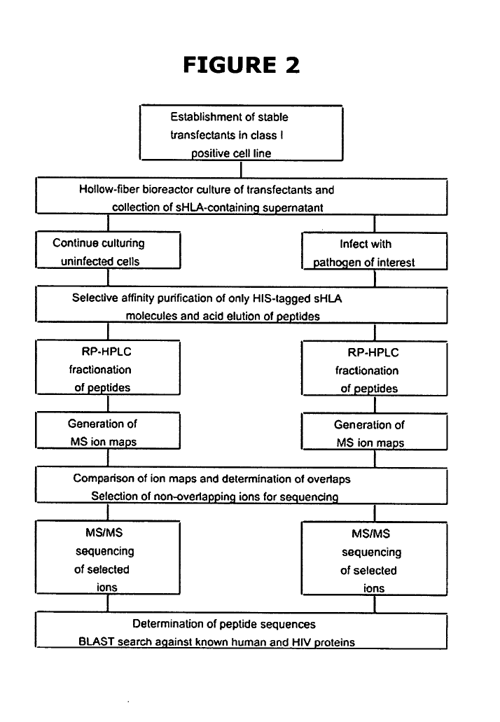

The presently disclosed and claimed invention, as well as the parent application U.S. Ser. No. 09/974,366, describe, in particular, peptide epitopes unique to HIV infected cells. Peptide epitopes unique to the HLA molecules of HIV infected cells were identified by direct comparison to HLA peptide epitopes from uninfected cells by the method illustrated in the flow chart of FIG. 2. Such method has been shown to be capable of identifying: (1) HLA presented peptide epitopes, derived from intracellular host proteins, that are unique to infected cells but not found on uninfected cells, and (2) that the intracellular source-proteins of the peptides are uniquely expressed/processed in HIV infected cells such that peptide fragments of the proteins can be presented by HLA on infected cells but not on uninfected cells.

The method of epitope discovery and comparative ligand mapping also, therefore, describes the unique expression of proteins in infected cells or, alternatively, the unique trafficking and processing of normally expressed host proteins such that peptide fragments thereof are presented by HLA molecules on infected cells. These HLA presented peptide fragments of intracellular proteins represent powerful alternatives for diagnosing virus infected cells and for targeting infected cells for destruction (i.e., vaccine development).

A group of the host source-proteins for HLA presented peptide epitopes unique to HIV infected cells represent source-proteins that are uniquely expressed in cancerous cells. For example, through using the methodology of the present invention a peptide fragment (SEQ ID NO:12) of reticulocalbin is uniquely found on HIV infected cells. A literature search indicates that the reticulocalbin gene is uniquely upregulated in cancer cells (breast cancer, liver cancer, colorectal cancer). Thus, the HLA presented peptide fragment of reticulocalbin which distinguishes HIV infected cells from uninfected cells can be inferred to also differentiate tumor cells from healthy non-tumor cells. Thus, HLA presented peptide fragments of host genes and gene products that distinguish the tumor cell and virus infected cell from healthy cells have been directly identified. The epitope discovery method is also capable of identifying host proteins that are uniquely expressed or uniquely processed on virus infected or tumor cells. HLA presented peptide fragments of such uniquely expressed or uniquely processed proteins can be used as vaccine epitopes and as diagnostic tools.

The methodology of targeting and detecting virus infected cells is not meant to target the virus-derived peptides. Rather, the methodology of the present invention indicates that the way to distinguish infected cells from healthy cells is through alterations in host encoded protein expression and processing. This is true for cancer as well as for virus infected cells. The methodology according to the present invention results in data which indicates, without reservation, that proteins/peptides distinguish virus/tumor cells from healthy cells.

In a brief example of the methodology of comparative ligand mapping utilized in the methods of the present invention, a cell line producing individual, soluble MHC molecules is constructed as described herein before and in U.S. Ser. No. 10/022,066. A portion of the transfected cell line is cocultured with a virus of interest, resulting in high-titre virus and providing infected cells. In the example provided herein and in detail in U.S. Ser. No. 10/022,066, the virus of interest is HIV. Alternatively, a portion of the cell line producing individual, soluble MHC molecules may be transformed to produce a tumor cell line.

The non-infected cell line and the cell line infected with HIV are both cultured in hollow-fiber bioreactors as described herein above and in detail in U.S. Ser. No. 10/022,066, and the soluble HLA-containing supernatant is then removed from the hollow-fiber bioreactors. The uninfected and infected harvested supernatants were then treated in an identical manner post-removal from the cell-pharm.

MHC class I-peptide complexes were affinity purified from the infected and uninfected supernatants using W6/32 antibody. Following elution, peptides were isolated from the class I molecules and separated by reverse phase HPLC fractionation. Separate but identical (down to the same buffer preparations) peptide purifications were done for each peptide-batch from uninfected and infected cells.

Fractionated peptides were then mapped by mass spectrometry to generate fraction-based ion maps. Spectra from the same fraction in uninfected/infected cells were manually aligned and visually assessed for the presence of differences in the ions represented by the spectra. Ions corresponding to the following categories were selected for MS/MS sequencing: (1) upregulation in infected cells (at least 1.5 fold over the same ion in uninfected cells), (2) downregulation in infected cells (at least 1.5 fold over the same ion in the uninfected cells), (3) presence of the ion only in infected cells, or (4) absence of ion in infected cells that is present in uninfected cells. In addition, multiple parameters were established before peptides were assigned to one of the above categories, including checking the peptide fractions preceding and following the peptide fraction by MS/MS to ensure that the peptide of interest was not present in an earlier or later fraction as well as generation of synthetic peptides and subjection to MS/MS to check for an exact match. In addition, one early quality control step involves examining the peptide's sequence to see if it fits the “predicted motif” defined by sequences that were previously shown to be presented by the MHC molecule utilized.

After identification of the epitopes, literature searches were performed on source proteins to determine their function within the infected cell, and the source proteins were classified into groups according to functions inside the cell. Secondly, source proteins were scanned for other possible epitopes which may be bound by other MHC class I alleles. Peptide binding predictions were employed to determine if other peptides presented from the source proteins were predicted to bind, and proteasomal prediction algorithms were likewise employed to determine the likelihood of a peptide being created by the proteasome.

In accordance with the present invention, Table I lists peptide ligands that have been identified as being presented by the B*0702 and A*0201 or B*1801 class I MHC molecule in cells infected with the HIV MN-1 virus but not in uninfected cells, and also lists one peptide ligand that has been identified as not being presented by the B*0702 class I MHC molecule in cells infected with the HIV MN-1 virus that is presented in uninfected cells. One of ordinary skill in the art can appreciate the novelty and usefulness of the present methodology in directly identifying such peptide ligands and the importance such identification has for numerous therapeutic (vaccine development, drug targeting) and diagnostic tools.

As stated above, Table I identifies the sequences of peptide ligands identified to date as being unique to HIV infected cells. Class I sHLA B*0702, A*0201 or B*1801 was harvested from T cells infected and not infected with HIV. Peptide ligands were eluted from B*0702, A*0201 or B*1801 and comparatively mapped on a mass spectrometer so that ions unique to infected cells were apparent. Ions unique to infected cells (and one ligand unique to uninfected cells) were subjected to mass spectrometric fragmentation for peptide sequencing.

TABLE I

Peptides Identified on Infected Cells that are not Present on Uninfected Cells
Restricting allele for Sequences marked with a (●) is HLA-B*0702.
Restricting allele for Sequences marked with a (▪) is HLA-A*0201 or HLA-B*1801.

| | | | | |
|---|---|---|---|---|
| | | | | Seq |
| | | Gene ID | | ID |
| Sequence | Source Protein | # | Category | No |

| | | | | | |
|---|---|---|---|---|---|
| ● | EQMFEDIISL | HIV MN-1, ENV | N/A | HIV-DERIVED | 1 |
| ● | IPCLLISFL | Cholinergic Receptor, alpha-3 | 1136 | Signal transduction; ion | 2 |
| | | polypeptide | | channel | |
| ● | STTAICATGL | Ubiquitin-specific protease 3 | 9960 | Ubiguitin-protease | 3 |
| | | | | activity; hydrolase | |
| | | | | activity | |
| ● | APAQNPEL | HLA-B associated transcript 3 (BAT3) | 7917 | MHC gene product | 4 |
| ● | LVMAPRTVL | HLA-B heavy chain leader segeunce | multiple | MHC gene product | 5 |
| ● | APFI[NS]PADX | Unknown, close to several cDNA's | N/A | UNKNOWN | 6 |
| ● | TPQSNRPVm | RNA polymerase II, polypeptide A | 5430 | DNA binding; protein | 7 |
| | | | | binding; transcription | |
| ● | AARPATSTL | Eukaryotic translation iniation | 1981 | RNA binding; translation | 8 |
| | | factor 4GI | | initiation factor | |
| ● | MAMMAALMA | Sparc-likek protein 1 | 13602 | calcium ion binding; | 9 |
| | | | | extracellular space | |
| ● | IATVDSYVI | Tenascin | 3371 | protein binding; | 10 |
| | | | | extracellular space | |
| ● | SPNQARAQAAL | Polypyrimidine tract binding | 5725 | RNA binding | 11 |
| | | protein 1 | | | |
| ● | GPRTAALGLL | Reticulocalbin 2 | 5955 | calcium ion binding; | 12 |
| | | | | protein binding | |
| ● | NPNQNKNVAL | ELAV (HuR) | 1994 | RNA binding; RNA | 13 |
| | | | | catabolism | |
| ● | RPYSNVSNL | Set-binding factor 1 | 6305 | protein phosphatase | 14 |
| | | | | activity | |
| ● | LPQANRDTL | Rac GTPase activating protein 1 | 29127 | electron transporter; | 15 |
| | | | | iron binding; | |
| | | | | intracellular signalling | |
| ● | QPRYPVNSV | TCP-1 alpha | 6950 | ATP binding; chaperone | 16 |
| | | | | activity | |
| ● | APAYSRAL | Heat shock protein 27 | 24471 | protein binding; | 17 |
| | | | | chaperone | |
| ● | APKRPPSAF | High mobility group protein 1 or 2 | 3164/ | DNA binding; DNA | 18 |
| | | | 3148 | unwinding | |
| ● | AASKERSGVSL | Histone H1 family member | multiple | DNA binding | 19 |
| ▪ | FIISRTQAL | karyopherin beta 2; importin beta 2; | 3842 | intracellular protein | 20 |
| | | transportin | | transport; nuclear | |
| | | | | import | |
| ▪ | SLAGSLRSV | FLJ00164 protein | 54436 | no description | 21 |
| ▪ | YGMPRQIL | similar to Homo sapiens mRNA for | 8407 | muscle development | 22 |
| | | KIAA0120 gene with GenBank Accession | | | |
| | | Number D21261.1 | | | |
| ▪ | MIIINKFV | hypothetical protein XP_103946 | unknown | no description | 23 |
| ▪ | ALWDIETGQQTV | G protein beta subunit | 2783 | GTPase activity; signal | 24 |
| | | | | transducer | |
| ▪ | VLMTEDIKL | eukaryotic translation initiation | 1981 | calcium ion binding; | 25 |
| | | factor 4 gamma, 1 | | extracellular space | |
| ▪ | YIYDKDMEII | usp22 | 23326 | Ubiquitin-protease | 26 |
| | | | | activity; hydrolase | |
| | | | | activity | |
| ▪ | ALMPVLNQV | homolog of yeast mRNA transport | 118460 | exosome constituent | 27 |
| | | regulator 3 | | | |
| ▪ | DLIIKGISV | TAR DNA binding protein | 23435 | RNA binding; | 28 |
| | | | | transcription factor | |
| | | | | activity | |
| ▪ | QLVDIIEKV | proteasome activator 28-gamma; 11S | 10197 | proteasome activator | 29 |
| | | regulator complex gamma subunit; | | activity | |
| | | proteasome activator subunit 3 | | | |
| | | isoform 2; Ki nuclear autoantigen | | | |
| ▪ | IMLEALERV | snRNP polypeptide G | 6637 | RNA binding; RNA | 30 |
| | | | | splicing; spliceosome | |
| | | | | assembly | |
| ▪ | DAYIRIVL | engulfment and cell motility 1 iso- | 9844 | signal transduction; | 31 |
| | | form 1; ced-12 homolog 1 | | cell motility | |
| ▪ | ILDPHVVLL | nucleoporin 88 kDa | 4927 | transporter activity; | 32 |
| | | | | nuclear pore transport | |
| ▪ | DAKIRIFDL | laminin receptor homolog or ribosomal | 6134 | ribosome constituent | 33 |
| | | protein L10 | | | |
| ▪ | ALLDKLYAL | brms2 or mitochondrial ribosomal | 55272 | RNA binding; ribosome | 34 |
| | | protein S4 or | | constituent | |
| ▪ | FMFDEKLVTV | serine/threonine protein phosphatase | 5537 | hydrolase activity; | 35 |
| | | catalytic subunit | | manganese ion binding | |
| ▪ | SLAQYLINV | hnRNP E2 | 5094 | DNA binding; RNA binding | 36 |
| ▪ | SLLQTLYKV | Similar to RAN GTPase activating | 5905 | GTPase activator | 37 |
| | | protein 1 | | activity; signal | |
| | | | | transducer | |
| ▪ | YMAELIERL | geminin | 51053 | cell cycle; DNA | 38 |
| | | | | replication inhibitor | |
| ▪ | FLYLIIISY | HIV-1 TAR RNA-binding protein B | unknown | no description | 39 |
| ▪ | SLLENLEKI | hnrnpC1/C2 | 3183 | MHC gene product | 40 |
| ▪ | FLFNKVVNL | yippee protein | 51646 | no description | 41 |
| ▪ | VLWDRTFSL | STAT-1 | 6772 | transcription factor | 42 |
| | | | | activity; signal | |
| | | | | transduction | |
| ▪ | SLASVFVRL | Similar to histone deacetylase 4 | 85009 | no description | 43 |
| ▪ | FLMDFIHQV | Nuclear pore complex protein Nup133 | 55746 | transporter activity; | 44 |
| | | (Nucleoporin Nup133) | | nuclear pore transport | |
| ▪ | FLWDEGFHQL | glucosidase I | 7841 | carbohydrate metabolism | 45 |
| ▪ | TALPRIFSL | TAP | 6890 | ABC transporter | 46 |
| ▪ | KLWEMDNMLI | T-cell activation protein | 93621 | ribosome constituent | 47 |
| ▪ | MVDGTLLLL | HLA-E leader sequence | 133015 | MHC gene product | 48 |
| ▪ | SLLDEFYKL | membrane component, chromosome 11, | 4076 | integral to plasma | 49 |
| | | surface marker 1 | | membrane | |
| ▪ | YLLPAIVHI | P68 RNA helicase | 1655 | ATP binding; RNA | 50 |
| | | | | binding; RNAhelicase | |
| | | | | activity | |
| ▪ | SLASLHPSV | PLAG-LIKE 1 or ZAC delta 2 protein or | 5325 | nucleic acid binding; | 51 |
| | | zinc finger protein or lost on trans- | | zinc ion binding | |
| | | formation LOT1 | | | |
| ▪ | KLWDIINVNI | steroid-dehydrogenase like | 83693 | oxidoreductase activity; | 52 |
| | | | | metabolism | |
| ▪ | KYPENFFLL | protein phosphatase I | 5500 | protein phosphatase | 53 |
| | | | | activity | |
| ▪ | YLLIEEDIRDLAA | TdT binding protein | 116092 | TdT binding | 54 |
| ▪ | DELQQPLEL | signal transducer and activator of | 6773 | transcription factor | 55 |
| | | transcription 2; signal transducer | | acivity; signal | |
| | | and activator of transcription 2, | | transduction | |
| | | 113kD; interferon alpha induced | | | |
| | | transcriptional activator | | | |
| ▪ | DEYEKLQVL | Dynein heavy chain, cytosolic (DYHC) | 1778 | ATP binding; nucleic | 56 |
| | | (Cytoplasmic dynein heavy chain 1) | | acid binding; mitotic | |
| | | (DHC1) | | spindle assembly | |
| ▪ | EEYQSLIRY | Protein CGI-126 (Protein HSPC155) | 51506 | ubiquitin-conjugating | 57 |
| | | | | enzyme activity | |
| ▪ | DDWKVIANY | c-myb protein | 4602 | DNA binding | 58 |
| ▪ | DELLNKFV | adaptor-related protein complex 2, | 160 | protein transporter | 59 |
| | | alpha 1 subunit isoform 1; adaptin, | | | |
| | | alpha A; clathrin-associated/assembly/ | | | |
| | | adaptor protein | | | |
| ▪ | DEFKVVVV | COPG protein | 22820 | vesicle coat complex | 60 |
| ▪ | LEGLTVVY | CGI-120 protein; likely ortholog of | 22818 | protein transporter | 61 |
| | | mouse coatomer protein complex, sub- | | activity | |
| | | unit zeta 1 | | | |
| ▪ | VEEILSVAY | RNA helicase II/Gu protein | 9188 | ATP binding; RNA binding | 62 |
| ▪ | DEDVLRYQF | cyclophilin 60 kDa; peptidylprolyl | 23759 | isomerase activity; | 63 |
| | | isomerase-like 2 isoform b; cyclo- | | protein folding | |
| | | philin-like protein CyP-60; peptidyl- | | | |
| | | prolyl cis-trans isomerase; | | | |
| ▪ | DEGTAFLVY | butyrylcholinesterase precursor | 590 | enzyme binding; | 64 |
| | | | | hydrolase activity | |
| ▪ | MEQVIFKY | ARP3 actin-related protein 3 homolog; | 10096 | constituent of | 65 |
| | | ARP3 (actin-related protein 3, yeast) | | cytoskeleton; cell | |
| | | homolog | | motility | |
| ▪ | NEQAFEEVF | replication protein A1, 70 kDa; rep- | 6117 | DNA binding; DNA | 66 |
| | | lication protein Al (70kD) | | recombination | |
| ▪ | VEEYVYEF | heat shock 105kD; heat shock 105kD | 10808 | ATP binding; chaperone | 67 |
| | | alpha; heat shock 105kD beta; heat | | activity | |
| | | shock 105 kDa protein 1 | | | |
| ▪ | DEIQVPVL | rab3-GAP regulatory domain | 25782 | GTPase activator; | 68 |
| | | | | intracellular protein | |
| | | | | transporter | |
| ▪ | DEYQFVERL | mitochondrial ribosomal protein L49; | 740 | structural constituent | 69 |
| | | neighbor of FAU; next to FAU [Homo | | of ribosomes | |
| | | sapiens] | | | |
| ▪ | DEYSIFPQTY | ras-related GTP-binding protein | 6009 | GTP binding; signal | 70 |
| | | | | tranducer | |
| ▪ | DEYSLVREL | talin | 7094 | actin binding; | 71 |
| | | | | cytoskeleton | |
| ▪ | EEVETFAF | HSP 90 | 3326 | chaperone activity | 72 |
| ▪ | NENDIRVMF | elav-type RNA-binding protein; | 10659 | RNA binding; RNA | 73 |
| | | RNA-binding protein BRUNOL3 | | processing | |
| ▪ | DEYDFYRSF | polymyositis/scleroderma autoantigen | 5394 | RNA binding; hydrolase | 74 |
| | | 2, 100 kDa; autoantigen PM-SCL; poly- | | activity | |
| | | myositis/scleroderma autoantigen 2 | | | |
| | | (100kD) | | | |
| ▪ | DEFQLLQAQY | AES-1 or AES-2 | 166 | transcription factor | 75 |
| | | | | activity | |
| ▪ | DEFEFLEKA | zinc finger protein 147 (estrogen- | 7706 | transcription factor | 76 |
| | | responsive finger protein) | | activity | |
| ▪ | DEMKVLVL | beta-fodrin | 6711 | actin binding | 77 |
| ▪ | DERVFVALY | similar to source of immunodominant MHC- | unknown | no description | 78 |
| | | associated peptides | | | |
| ▪ | IENPFGETF | integral inner nuclear membrane protein | 23592 | integral to inner | 79 |
| | | | | nuclear membrane | |
| ▪ | SEFELLRSY | sorting nexin 4 | 8723 | protein transporter: | 80 |
| | | | | intracellular signalling | |
| ▪ | DEGRLVLEF | Acyl-coA/cholesterol acyltransferase | 6646 | no description | 81 |
| ▪ | DEGWFLIL | RNA helicase family | 10973 | ATP binding; nucleic | 82 |
| | | | | acid binding; hydrolase | |
| | | | | activity | |
| ▪ | DEISFVNF | structure specific recognition | 6749 | DNA binding; | 83 |
| | | protein 1; recombination signal | | transcription regulator | |
| | | sequence recognition protein; | | activity | |
| | | chromatin-specific transcription | | | |
| | | elongation factor 80 kDa subunit | | | |
| ▪ | SEVLSWQF | signal transducer and activator of | 6772 | transcription factor | 84 |
| | | transcription-1; | activity; signal | | |
| | | | | transduction | |
| ▪ | YEILLGKATLY | T cell receptor beta-chain | 6957 | MHC binding; receptor | 85 |
| | | | | activity | |
| ▪ | YENLLAVAF | unnamed protein product | 79739 | protein modification | 86 |
| ▪ | DETQIFSYF | nucleolar phosphoprotein Nopp34 | 84365 | RNA binding; protein | 87 |
| | | | | binding | |
| ▪ | MEPLRVLEL | DNA methyltransferase 2 isoform d; | 1787 | DNA binding; DNA | 88 |
| | | DNA methyltransferase-2; DNA methyl- | | methylation | |
| | | transferase homolog HsaIIP; DNA MTase | | | |
| | | homolog HsaIIP | | | |
| ▪ | MPLGKTLPC | laminin | 3918 | protein binding; | 89 |
| | | | | structural molecule | |
| | | | | activity | |
| ▪ | VYMDWYEKF | U5 snrnp 200 kDa helicase | 23020 | ATP binding; nucleic | 90 |
| | | | | acid binding; RNA | |
| | | | | splicing | |
| ▪ | SELLIHVF | protein kinase c-iota | 5584 | ATP binding; protein | 91 |
| | | | | binding | |
| ▪ | DEHLITFF | U5 snrnp 200 kDa helicase | 23020 | ATP binding; nucleic | 92 |
| | | | | acid binding; RNA | |
| | | | | splicing | |
| ▪ | DEFKIGELF | DNA-PKcs | 5591 | DNA binding; transferase | 93 |
| | | | | activity | |
| ▪ | DELEIIEGMKF | (Heat shock protein 60) (HSP-60) | 3329 | ATP binding; chaperone | 94 |
| | | | | activity | |
| ▪ | KYLLSATKLR | melanoma-derived leucine zipper, | 56169 | no description | 95 |
| | | extra-nuclear factor | | | |
| ▪ | SEIELFRVF | U5 small nuclear ribonucleoprotein | 23020 | ATP binding; nucleic | 96 |
| | | 200 kDa helicase | | acid binding; RNA | |
| | | | | splicing | |
| ▪ | LEDVLPLAF | HP1-BP74 | 50809 | DNA binding; nucleosome | 97 |
| | | | | assembly | |

In order to provide an analysis of peptides after HIV-infection under as-close-as possible conditions as those that would occur inside an infected person, a human T cell line was utilized for infection with HIV. This cell line, Sup-T1, possesses its own class I; HLA-A and -B types are A*2402, A*6801, B*0801, and B*1801. Although only the soluble class I specifically introduced into the cell should be secreted, under some conditions shedding of full-length class I molecules has been observed. It is believed that HLA-B*1801 is shed after HIV infection.

Analysis of soluble A*0201 produced a number of ligands that did not appear to fit the A*0201 peptide motif (an indication of which amino acids are preferred at particular positions of the peptide). For instance, A*0201 prefers peptides with an L at position 2 (P2) and an L or V at P9. Most of the peptides that did not match the A*0201 motif had an E at P2 and a Y or F at P9.

Upon inspection, these peptides were most likely derived from B*1801. To confirm, several peptides from B*1801 molecules in a class I negative cell line were sequenced, and several overlapping peptides were identified. Therefore, at this point, the peptides are labeled as either A*0201 or B*1801 restricted. Tests are currently being performed to delineate which of the two molecules binds each peptide. However, simple analysis of the peptide sequence (P2 and P9 amino acids) should be sufficient to determine the restricting molecule, and such simple analysis is within the ability of a person having ordinary skill in the art.

The methodology used herein is to use sHLA to determine what is unique to unhealthy cells as compared to healthy cells. Using sHLA to survey the contents of a cell provides a look at what is unique to unhealthy cells in terms of proteins that are processed into peptides. The data summarized in TABLE I shows that the epitope discovery technique described herein is capable of identifying sHLA bound epitopes and their corresponding source proteins which are unique to infected/unhealthy cells.

Likewise, peptide ligands presented in individual class I MHC molecules in an uninfected cell that are not presented by individual class I MHC molecules in an uninfected cell can also be identified. The peptide “GSHSMRY” (SEQ ID NO:98), for example, was identified by the method of the present invention as being an individual class I MHC molecule which is presented in an uninfected cell but not in an infected cell. The source protein for this peptide is MHC Class I Heavy Chain, which could be derived from multiple alleles, i.e., HLA-B*0702 or HLA-G, etc.

The utility of this data is at least threefold. First, the data indicates what comes out of the cell with HLA. Such data can be used to target CTL to unhealthy cells. Second, antibodies can be targeted to specifically recognize HLA molecules carrying the ligand described. Third, realization of the source protein can lead to therapies and diagnostics which target the source protein. Thus, an epitope unique to unhealthy cells also indicates that the source protein is unique in the unhealthy cell.

The methods of epitope discovery and comparative ligand mapping described herein are not limited to cells infected by a microorganism such as HIV. Unhealthy cells analyzed by the epitope discovery process described herein can arise from virus infection or also from cancerous transformation. Unhealthy cells may also be produced following treatment of healthy cells with a cancer causing agent, such as but not limited to, nicotine, or by a disease state cytokine such as IL-4. In addition, the status of an unhealthy cell can also be mimicked by transfecting a particular gene known to be expressed during viral infection or tumor formation. For example, particular genes of HIV can be expressed in a cell line as described (Achour, A., et al., AIDS Res Hum Retroviruses, 1994. 10(1): p. 19-25; and Chiba, M., et al., CTL. Arch Virol, 1999. 144(8): p. 1469-85, all of which are expressly incorporated herein by reference) and then the epitope discovery process performed to identify how the expression of the transferred gene modifies epitope presentation by sHLA. In a similar fashion, genes known to be upregulated during cancer (Smith, E. S., et al., Nat Med, 2001. 7(8): p. 967-72, which is expressly incorporated herein by reference) can be transferred in cells with sHLA and epitope discovery then completed. Thus, epitope discovery with sHLA as described herein can be completed on cells infected with intact pathogens, cancerous cells or cell lines, or cells into which a particular cancer, viral, or bacterial gene has been transferred. In all these instances the sHLA described here will provide a means for detecting what changes in terms of epitope presentation and the source proteins for the epitopes.

Thus, in accordance with the present invention, there has been provided a method of epitope discovery and comparative ligand mapping that includes methodology for producing and manipulating Class I and Class II MHC molecules from gDNA as well as methodology for directly discovering epitopes unique to infected or tumor cells that fully satisfies the objectives and advantages set forth herein above. Although the invention has been described in conjunction with the specific drawings, experimentation, results and language set forth herein above, it is evident that many alternatives, modifications, and variations will be apparent to those skilled in the art. Accordingly, it is intended to embrace all such alternatives, modifications and variations that fall within the spirit and broad scope of the invention.

1. A peptide ligand for an individual class I molecule isolated by a method comprising the steps of:
providing a cell line containing a construct that encodes an individual soluble class I molecule, the cell line being able to naturally process proteins into peptide ligands capable of being loaded into antigen binding grooves of class I molecules;
culturing the cell line under conditions which allow for expression of the individual soluble class I molecules from the construct, such conditions also allowing for endogenous loading of a peptide ligand into the antigen binding groove of each individual soluble class I molecule prior to secretion of the individual soluble class I molecules from the cell;
isolating the secreted individual soluble class I molecules having the endogenously loaded peptide ligands bound thereto; and
separating the peptide ligands from the individual soluble class I molecules, wherein at least one of the peptide ligands comprises at least one of SEQ ID NOS:14-97.

2. A peptide ligand for an individual class I molecule comprising at least one of SEQ ID NOS:14-97.

3. An endogenously loaded peptide ligand presented by an individual class I molecule on an infected cell but not on an uninfected cell, wherein the peptide ligand comprises at least one of SEQ ID NOS:14-97.

4. A peptide ligand presented by an individual class I molecule on an infected cell but not on an uninfected cell, the peptide ligand identified by a method comprising the steps of:
providing an uninfected cell line containing a construct that encodes an individual soluble class I molecule, the cell line being able to naturally process proteins into peptide ligands capable of being loaded into antigen binding grooves of class I molecules;
infecting a portion of the uninfected cell line with at least one of a microorganism, a gene from a microorganism or a tumor gene, thereby providing an infected cell line;
culturing the uninfected cell line and the infected cell line under conditions which allow for expression of the individual soluble class I molecules from the construct, such conditions also allowing for endogenous loading of a peptide ligand in the antigen binding groove of each individual soluble class I molecule prior to secretion of the individual soluble class I molecules from the cell;
isolating the secreted individual soluble class I molecules having the endogenously loaded peptide ligands bound thereto from the uninfected cell line and the infected cell line;
separating the endogenously loaded peptide ligands from the individual soluble class I molecules from the uninfected cell and the endogenously loaded peptide ligands from the individual soluble class I molecules from the infected cell;
isolating the endogenously loaded peptide ligands from the uninfected cell line and the endogenously loaded peptide ligands from the infected cell line;
comparing the endogenously loaded peptide ligands isolated from the infected cell line to the endogenously loaded peptide ligands isolated from the uninfected cell line; and
identifying at least one endogenously loaded peptide ligand presented by the individual soluble class I molecule on the infected cell line that is not presented by the individual soluble class I molecule on the uninfected cell line, wherein the at least one endogenously loaded peptide ligand comprises at least one of SEQ ID NOS:14-97.

5. The peptide ligand of claim 4 wherein, in the step of providing an uninfected cell line containing a construct that encodes an individual soluble class I molecule, the uninfected cell line containing the construct that encodes the individual soluble class I molecule is produced by a method comprising the steps of:
obtaining genomic DNA or cDNA encoding at least one class I molecule;
identifying an allele encoding an individual class I molecule in the genomic DNA or cDNA;
PCR amplifying the allele encoding the individual class I molecule in a locus specific manner such that a PCR product produced therefrom encodes a truncated, soluble form of the individual class I molecule;
cloning the PCR product into an expression vector, thereby forming a construct that encodes the individual soluble class I molecule; and
transfecting the construct into an uninfected cell line.

6. The peptide ligand of claim 5, wherein the construct further encodes a tag which is attached to the individual soluble class I molecule and aids in isolating the individual soluble class I molecule.

7. The peptide ligand of claim 6, wherein the tag is selected from the group consisting of a HIS tail and a FLAG tail.

8. The peptide ligand of claim 6, wherein the tag is encoded by a PCR primer utilized in the step of PCR amplifying the allele encoding the individual class I molecule.

9. The peptide ligand of claim 6, wherein the tag is encoded by the expression vector into which the PCR product is cloned.

10. The peptide ligand of claim 4, wherein the at least one endogenously loaded peptide ligand is obtained from a protein encoded by at least one of the microorganism, the gene from the microorganism or the tumor gene with which the portion of the uninfected cell line is infected to form the infected cell line.

11. The peptide ligand of claim 4, wherein the at least one endogenously loaded peptide ligand is obtained from a protein encoded by the uninfected cell line.

12. The peptide ligand of claim 4, wherein the portion of the uninfected cell line is infected with HIV.


SEQUENCE LISTING (98 sequences)

SEQ ID NO: 1 - protein, 10 aa, Homo sapiens
EQMFEDIISL

SEQ ID NO: 2 - protein, 9 aa, Homo sapiens
IPCLLISFL

SEQ ID NO: 3 - protein, 10 aa, Homo sapiens
STTAICATGL

SEQ ID NO: 4 - protein, 8 aa, Homo sapiens
APAQNPEL

SEQ ID NO: 5 - protein, 9 aa, Homo sapiens
LVMAPRTVL

SEQ ID NO: 6 - protein, 9 aa, Homo sapiens
  MISC_FEATURE (5)..(5): N or S
APFIXPADX

SEQ ID NO: 7 - protein, 9 aa, Homo sapiens
TPQSNRPVM

SEQ ID NO: 8 - protein, 9 aa, Homo sapiens
AARPATSTL

SEQ ID NO: 9 - protein, 9 aa, Homo sapiens
MAMMAALMA

SEQ ID NO: 10 - protein, 9 aa, Homo sapiens
IATVDSYVI

SEQ ID NO: 11 - protein, 11 aa, Homo sapiens
SPNQARAQAAL

SEQ ID NO: 12 - protein, 10 aa, Homo sapiens
GPRTAALGLL

SEQ ID NO: 13 - protein, 10 aa, Homo sapiens
NPNQNKNVAL

SEQ ID NO: 14 - protein, 9 aa, Homo sapiens
RPYSNVSNL

SEQ ID NO: 15 - protein, 9 aa, Homo sapiens
LPQANRDTL

SEQ ID NO: 16 - protein, 9 aa, Homo sapiens
QPRYPVNSV

SEQ ID NO: 17 - protein, 8 aa, Homo sapiens
APAYSRAL

SEQ ID NO: 18 - protein, 9 aa, Homo sapiens
APKRPPSAF

SEQ ID NO: 19 - protein, 11 aa, Homo sapiens
AASKERSGVSL

SEQ ID NO: 20 - protein, 9 aa, Homo sapiens
FIISRTQAL

SEQ ID NO: 21 - protein, 9 aa, Homo sapiens
SLAGSLRSV

SEQ ID NO: 22 - protein, 8 aa, Homo sapiens
YGMPRQIL

SEQ ID NO: 23 - protein, 8 aa, Homo sapiens
MIIINKFV

SEQ ID NO: 24 - protein, 12 aa, Homo sapiens
ALWDIETGQQTV

SEQ ID NO: 25 - protein, 9 aa, Homo sapiens
VLMTEDIKL

SEQ ID NO: 26 - protein, 10 aa, Homo sapiens
YIYDKDMEII

SEQ ID NO: 27 - protein, 9 aa, Homo sapiens
ALMPVLNQV

SEQ ID NO: 28 - protein, 9 aa, Homo sapiens
DLIIKGISV

SEQ ID NO: 29 - protein, 9 aa, Homo sapiens
QLVDIIEKV

SEQ ID NO: 30 - protein, 9 aa, Homo sapiens
IMLEALERV

SEQ ID NO: 31 - protein, 8 aa, Homo sapiens
DAYIRIVL

SEQ ID NO: 32 - protein, 9 aa, Homo sapiens
ILDPHVVLL

SEQ ID NO: 33 - protein, 9 aa, Homo sapiens
DAKIRIFDL

SEQ ID NO: 34 - protein, 9 aa, Homo sapiens
ALLDKLYAL

SEQ ID NO: 35 - protein, 10 aa, Homo sapiens
FMFDEKLVTV

SEQ ID NO: 36 - protein, 9 aa, Homo sapiens
SLAQYLINV

SEQ ID NO: 37 - protein, 9 aa, Homo sapiens
SLLQTLYKV

SEQ ID NO: 38 - protein, 9 aa, Homo sapiens
YMAELIERL

SEQ ID NO: 39 - protein, 9 aa, Homo sapiens
FLYLIIISY

SEQ ID NO: 40 - protein, 9 aa, Homo sapiens
SLLENLEKI

SEQ ID NO: 41 - protein, 9 aa, Homo sapiens
FLFNKVVNL

SEQ ID NO: 42 - protein, 9 aa, Homo sapiens
VLWDRTFSL

SEQ ID NO: 43 - protein, 9 aa, Homo sapiens
SLASVFVRL

SEQ ID NO: 44 - protein, 9 aa, Homo sapiens
FLMDFIHQV

SEQ ID NO: 45 - protein, 10 aa, Homo sapiens
FLWDEGFHQL

SEQ ID NO: 46 - protein, 9 aa, Homo sapiens
TALPRIFSL

SEQ ID NO: 47 - protein, 10 aa, Homo sapiens
KLWEMDNMLI

SEQ ID NO: 48 - protein, 9 aa, Homo sapiens
MVDGTLLLL

SEQ ID NO: 49 - protein, 9 aa, Homo sapiens
SLASLHPSV

SEQ ID NO: 50 - protein, 9 aa, Homo sapiens
YLLPAIVHI

SEQ ID NO: 51 - protein, 9 aa, Homo sapiens
SLASLHPSV

SEQ ID NO: 52 - protein, 10 aa, Homo sapiens
KLWDIINVNI

SEQ ID NO: 53 - protein, 9 aa, Homo sapiens
KYPENFFLL

SEQ ID NO: 54 - protein, 13 aa, Homo sapiens
YLLIEEDIRDLAA

SEQ ID NO: 55 - protein, 9 aa, Homo sapiens
DELQQPLEL

SEQ ID NO: 56 - protein, 9 aa, Homo sapiens
DEYEKLQVL

SEQ ID NO: 57 - protein, 9 aa, Homo sapiens
EEYQSLIRY

SEQ ID NO: 58 - protein, 9 aa, Homo sapiens
DDWKVIANY

SEQ ID NO: 59 - protein, 8 aa, Homo sapiens
DELLNKFV

SEQ ID NO: 60 - protein, 8 aa, Homo sapiens
DEFKVVVV

SEQ ID NO: 61 - protein, 8 aa, Homo sapiens
LEGLTVVY

SEQ ID NO: 62 - protein, 9 aa, Homo sapiens
VEEILSVAY

SEQ ID NO: 63 - protein, 9 aa, Homo sapiens
DEDVLRYQF

SEQ ID NO: 64 - protein, 9 aa, Homo sapiens
DEGTAFLVY

SEQ ID NO: 65 - protein, 8 aa, Homo sapiens
MEQVIFKY

SEQ ID NO: 66 - protein, 9 aa, Homo sapiens
NEQAFEEVF

SEQ ID NO: 67 - protein, 8 aa, Homo sapiens
VEEYVYEF

SEQ ID NO: 68 - protein, 8 aa, Homo sapiens
DEIQVPVL

SEQ ID NO: 69 - protein, 9 aa, Homo sapiens
DEYQFVERL

SEQ ID NO: 70 - protein, 10 aa, Homo sapiens
DEYSIFPQTY

SEQ ID NO: 71 - protein, 9 aa, Homo sapiens
DEYSLVREL

SEQ ID NO: 72 - protein, 8 aa, Homo sapiens
EEVETFAF

SEQ ID NO: 73 - protein, 9 aa, Homo sapiens
NENDIRVMF

SEQ ID NO: 74 - protein, 9 aa, Homo sapiens
DEYDFYRSF

SEQ ID NO: 75 - protein, 10 aa, Homo sapiens
DEFQLLQAQY

SEQ ID NO: 76 - protein, 9 aa, Homo sapiens
DEFEFLEKA

SEQ ID NO: 77 - protein, 8 aa, Homo sapiens
DEMKVLVL

SEQ ID NO: 78 - protein, 9 aa, Homo sapiens
DERVFVALY

SEQ ID NO: 79 - protein, 9 aa, Homo sapiens
IENPFGETF

SEQ ID NO: 80 - protein, 9 aa, Homo sapiens
SEFELLRSY

SEQ ID NO: 81 - protein, 9 aa, Homo sapiens
DEGRLVLEF

SEQ ID NO: 82 - protein, 8 aa, Homo sapiens
DEGWFLIL

SEQ ID NO: 83 - protein, 8 aa, Homo sapiens
DEISFVNF

SEQ ID NO: 84 - protein, 8 aa, Homo sapiens
SEVLSWQF

SEQ ID NO: 85 - protein, 11 aa, Homo sapiens
YEILLGKATLY

SEQ ID NO: 86 - protein, 9 aa, Homo sapiens
YENLLAVAF

SEQ ID NO: 87 - protein, 9 aa, Homo sapiens
DETQIFSYF

SEQ ID NO: 88 - protein, 9 aa, Homo sapiens
MEPLRVLEL

SEQ ID NO: 89 - protein, 9 aa, Homo sapiens
MPLGKTLPC

SEQ ID NO: 90 - protein, 9 aa, Homo sapiens
VYMDWYEKF

SEQ ID NO: 91 - protein, 8 aa, Homo sapiens
SELLIHVF

SEQ ID NO: 92 - protein, 8 aa, Homo sapiens
DEHLITFF

SEQ ID NO: 93 - protein, 9 aa, Homo sapiens
DEFKIGELF

SEQ ID NO: 94 - protein, 11 aa, Homo sapiens
DELEIIEGMKF

SEQ ID NO: 95 - protein, 10 aa, Homo sapiens
KYLLSATKLR

SEQ ID NO: 96 - protein, 9 aa, Homo sapiens
SEIELFRVF

SEQ ID NO: 97 - protein, 9 aa, Homo sapiens
LEDVLPLAF

SEQ ID NO: 98 - protein, 7 aa, Homo sapiens
GSHSMRY